(19) United States
(12) Patent Application Publication
(10) Pub. No.: US 2018/0154980 A1
(43) Pub. Date: Jun. 7, 2018

(54) CONTROL DEVICE FOR AN ELECTRIC BICYCLE

(71) Applicant: Wen-Sung Lee, Taichung City (TW)

(72) Inventor: Wen-Sung Lee, Taichung City (TW)

(21) Appl. No.: 15/368,694

(22) Filed: Dec. 5, 2016

(51) Int. Cl. B62M 6/50; B62K 11/10; B62M 6/90; B62J 99/00; B60L 11/18; H04B 1/38; A61B 5/22; A61B 5/00; A61B 5/024

(52) U.S. Cl. CPC B62M 6/50; B62K 11/10; B62M 6/90; B62J 99/00; B60L 11/1851; H04B 1/38; B60L 2220/44; A61B 5/0004; A61B 5/02438; A61B 5/6895; A61B 5/6898; B62J 2099/0006; B60L 2220/12; A61B 5/222

(57) ABSTRACT

A control device for an electric bicycle includes a motor, a battery, a micro controller, an acceleration detector and a transmitter electrically connected to each other. A physical-condition monitor detects the rider's heart rate signal. A computer calculates an electric power consumption value from the micro controller, an acceleration value from the acceleration detector, and the heart rate signal to create a motor output adjustment signal which is created according to the practical conditions of the rider. The motor output adjustment signal is sent to the micro controller to adjust the output of the battery to the motor and the assistance force to the bicycle.

### BACKGROUND OF THE INVENTION

### 1. Fields of the Invention

The present invention relates to an electric bicycle, and more particularly, to a control device for an electric bicycle.

### 2. Descriptions of Related Art

One of the conventional electric bicycles including power assistance electric bicycles and pure electric bicycles known to applicant includes a control device which is used for an electric bicycle with a speed adjustable bicycle. The control device has a computer and a treading speed detector which is electrically connected to the computer. The treading speed detector detects the treading speed and sends the detected information to the computer. A speed detector is electrically connected to the computer and detects the speed of the bicycle and sends the detected information to the computer. A gear-shifting source is electrically connected to the computer and provides the current gear to the computer. A data base is electrically connected to the computer so as to provide pre-set values to the computer such that the computer compares the current values of the treading speed, the moving speed, the gear and the ratio of gears with the pre-set values in the data base so as to make judgments. When the first value of the ratio between the moving speed and the ratio of gears and the second value of the treading speed are both smaller than the pre-set value which is the value under normal operational status, the computer judges that the bicycle is operated at low speed of treading, so that a larger assistance force is output to the bicycle. When the first and second values are medium compared with the pre-set value, a medium assistance force is output to the bicycle. When the first and second values are larger than with the pre-set value, a small assistance force is output to the bicycle. If the three conditions are not detected, no assistance force is output to the bicycle.

However, the first and second values involve only three factors which are not sufficient to precisely judge the real operation of a rider operating a bicycle. Therefore, the assistance force output to the bicycle does not meet the practical needs.

The present invention intends to provide a control device for an electric bicycle, and the control device is cooperated with a motor, a battery, a micro controller, an acceleration detector and a transmitter so as to provide the assistance force that meets the real needs when different riders operate the electric bicycle.

### SUMMARY OF THE INVENTION

The present invention relates to a control device for an electric bicycle and comprises a motor, a battery, a micro controller, an acceleration detector and a transmitter electrically connected to each other. The micro controller controls the battery to provide power to the motor of the electric bicycle, and monitors the power output from the battery and defines an electric power consumption value. The acceleration detector detects the acceleration speed of the bicycle and creates an acceleration value. The transmitter transmits the electric power consumption value and the acceleration value to a computer. The computer has a wireless receiving/emitting unit and a force assistance unit, wherein the wireless receiving/emitting unit receives the electric power consumption value and the acceleration value. The force assistance unit calculates the electric power consumption value and the acceleration value, and creates a motor output adjustment signal. The wireless receiving/emitting unit and the transmitter send the motor output adjustment signal to the micro controller which adjusts the battery to output power to the motor so as to change assistance force to the rider who operates the electric bicycle.

Preferably, a physical-condition monitor is attached to the rider, the physical-condition monitor is adapted to monitor physical condition of the rider and creating a heart rate signal which is sent to the computer wirelessly. The force assistance unit calculates the electric power consumption value, the acceleration value and the heart rate signal and creates the motor output adjustment signal. The wireless receiving/emitting unit and the transmitter send the motor output adjustment signal to the micro controller which adjusts the battery to output power to the motor so as to change or adjust assistance force to the rider who operates the electric bicycle.

Preferably, the computer is a laptop, a smart phone or a tablet computer, and includes a Global Positioning System navigation chip (GPS navigation chip), a touch screen, a memory unit and a setting unit. The GPS navigation chip receives signals via satellites to obtain current location and altitude information. The setting unit sets a travel route, and the touch screen displays the travel route and current locations. The memory unit records the electric power consumption value, the acceleration value and the heart rate signal from a start point of the travel route. The force assistance unit calculates the altitude from the GPS navigation chip and calculates inclination of the travel route, and the force assistance unit calculates the electric power consumption value, the acceleration value and the heart rate signal to create the motor output adjustment signal.

Preferably, the acceleration detector is a gyro.

Preferably, the physical-condition monitor supports a wireless ring or a heart rate band.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a side view to show the electric bicycle with the control device of the present invention;

FIG. 2 illustrates a flow chart of the parts of the control device of the present invention, and

FIG. 3 shows a rider operates the electric bicycle with the control device of the present invention.

### DETAILED DESCRIPTION OF THE PREFERRED EMBODIMENT

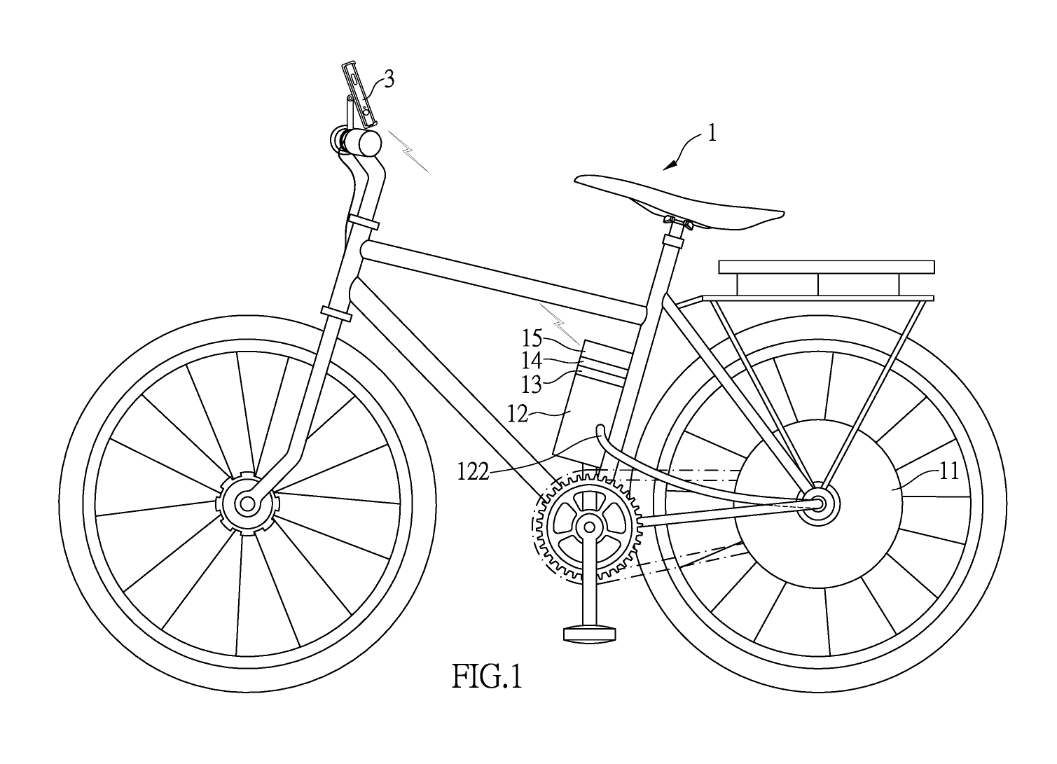

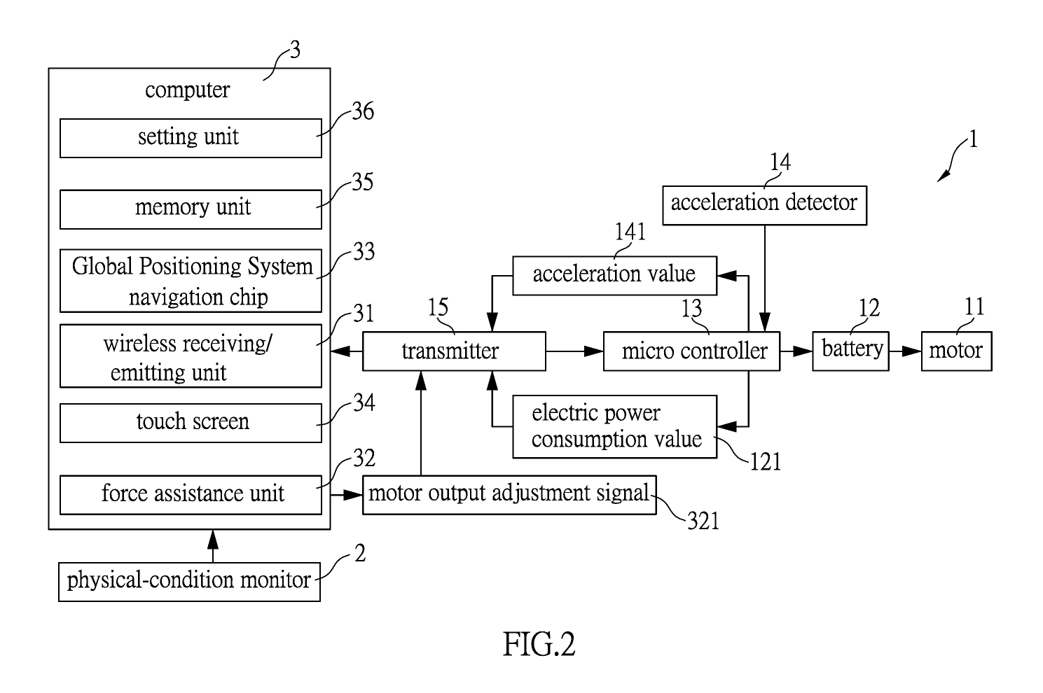

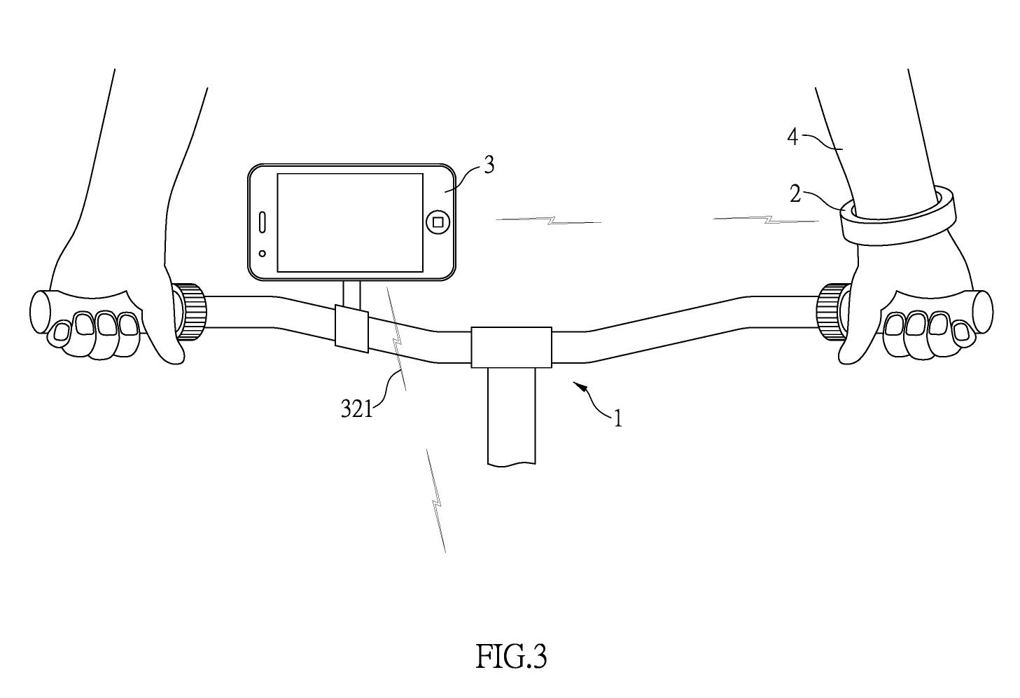

Referring to FIGS. 1 to 3, the control device for an electric bicycle 1 of the present invention comprises a motor 11, a battery 12, a micro controller 13, an acceleration detector 14 and a transmitter 15 electrically connected to each other. The micro controller 13 controls the battery 12 to provide power to the motor 11 of the electric bicycle 1, and monitors the power output from the battery 12 and defines an electric power consumption value 121. The battery 12 is electrically connected to the motor 11 by wires 122. The acceleration detector 14 detects the acceleration speed of the bicycle 1 and creates an acceleration value 141. The transmitter 15 transmits the electric power consumption value 121 and the acceleration value 141 to a computer 3.

The computer 3 has a wireless receiving/emitting unit 31 and a force assistance unit 32, wherein the wireless receiving/emitting unit 31 receives the electric power consumption value 121 and the acceleration value 141, and the force assistance unit 32 calculates the electric power consumption value 121 and the acceleration value 141, and creates a motor output adjustment signal 321. The wireless receiving/emitting unit 31 and the transmitter 15 send the motor output adjustment signal 321 to the micro controller 13 which adjusts the battery 12 to output power to the motor 11 so as to change or adjust the assistance force supplied to the rider 4 who operates the electric bicycle 1.

A physical-condition monitor 2 is attached to the rider 4 and monitors physical condition of the rider 4 so as to create a heart rate signal 21 which is sent to the computer 3 wirelessly. The force assistance unit 32 calculates the electric power consumption value 121, the acceleration value 141 and the heart rate signal 121 and creates the motor output adjustment signal 321. The wireless receiving/emitting unit 31 and the transmitter 15 send the motor output adjustment signal 321 to the micro controller 13 which adjusts the battery 12 to output power to the motor 11 so as to change or adjust the assistance force to the rider 4 who operates the electric bicycle 1. In this embodiment, the acceleration detector 14 is a gyro. The physical-condition monitor 2 supports a wireless ring or a heart rate band. The computer 3 is a laptop, a smart phone or a tablet computer, and includes a Global Positioning System navigation chip 33 (GPS navigation chip), a touch screen 34, a memory unit 35 and a setting unit 36. The GPS navigation chip 33 receives signals via satellites to obtain the current location and altitude information. The setting unit 36 sets a travel route, and the touch screen 34 displays the travel route and current locations. The memory unit 35 records the electric power consumption value 121, the acceleration value 141 and the heart rate signal 21 from the start point of the travel route. The force assistance unit 32 calculates the altitude from the GPS navigation chip 33 and calculates inclination of the travel route, and the force assistance unit 32 calculates the electric power consumption value 121, the acceleration value 141 and the heart rate signal 21 to create the motor output adjustment signal 321.

The rider 4 has to set the physical-condition monitor 2, the transmitter 15, the wireless receiving/emitting unit 31 to be wirelessly paired to each other. Then the travel route is set by the GPS navigation chip 33 and the setting unit 36. The touch screen 34 displays the necessary information such as the travel route and the current locations. The memory unit 35 records the electric power consumption value 121, the acceleration value 141 and the heart rate signal 21 from the start point of the travel route. The mileage and the time used are also recorded. The GPS navigation chip 33 receives signals via satellites to obtain the current location and altitude information. The force assistance unit 32 calculates the altitude from the GPS navigation chip 33 and calculates the inclination of the travel route. The force assistance unit 32 also calculates the electric power consumption value 121, the acceleration value 141 and the heart rate signal 21 to create the motor output adjustment signal 321. The wireless receiving/emitting unit 31 and the transmitter 15 send the motor output adjustment signal 321 to the micro controller 13 which adjusts the battery 12 to output power to the motor 11 so as to change or adjust the assistance force to the rider 4 who operates the electric bicycle 1.

The force assistance unit 32 creates the motor output adjustment signal 321 by the relationship between the force output from the rider 4 and the assistance force from the motor 11. When a force applies to an object, the kinetic energy increases from E_{K0} to E_{K} and the work W=ΔE_{K}=E_{K}−E_{K0}=Fd.

For the linear movement of the change of speed,

$$Fd = mad = mad = ma ( \frac{{v}_{2}^{2} - {v}_{1}^{2}}{za} ) = \frac{{mv}_{2}^{2}}{2} - \frac{{mv}_{1}^{2}}{2} = Δ {E}_{K} , \\ d = ( \frac{{v}_{2}^{2} - {v}_{1}^{2}}{2 a} ) ,$$

v₂²=v₁²+2ad, wherein “m” is the mass, v₁ and v₂ respectively represent the initial speed and the terminal speed of the mass. The acceleration speed is represented by “a”. In a general situation,

$$\begin{matrix}W = {\int }_{t 1}^{t 2} F \cdot vdt \\ = {\int }_{t 1}^{t 2} Fvdt \\ = {\int }_{t 1}^{t 2} madt \\ = m {\int }_{t 1}^{t 2} v \frac{dv}{dt} dt \\ = m {\int }_{t 1}^{t 2} vdv \\ = \frac{1}{2} m ( {v}_{2}^{2} - {v}_{2}^{2} )\end{matrix}$$

In this embodiment, “a” represents the acceleration speed detected on the Y-axis by the micro controller 13. The equation is a=hpa+mpa, wherein mpa represents the output motor power from the motor 11, and the hpa represents the human power, that is to say, hpa=a−mpa. The acceleration speed of the bicycle is represented by force that the rider 4 applies to the bicycle, so that the acceleration speed detected on the Y-axis by the micro controller. 13 is used as the calculation factor such that the acceleration speed caused by the rider is obtained.

The force assistance unit 32 of the computer 3 displays the hₚαmₚa by a specific curve or figure and the motor power is adjusted according to the change of the hpa. In other words, the curve represented the motor output formed by the mpa is adjusted to generate the motor output adjustment signal 321 that is purely caused by the rider 4 (human power). The micro controller 13 adjusts the battery 12 to output power to the motor 11 according to the motor output adjustment signal 321 to provide proper assistance force to the bicycle 1.

The output of the battery 12 is adjusted according to the inclination to ensure that the power from the battery 12 is sufficient to let the rider 4 to operate the bicycle 1 to its destination by the results that the force assistance unit 32 calculates the altitude from the GPS navigation chip 33 and calculates the inclination of the travel route, and the force assistance unit 32 calculates the electric power consumption value 121, the acceleration value 141 and the heart rate signal 21 to create the motor output adjustment signal 321.

In addition, the computer 3 is able to check the physical condition of the rider 4 by comparison of the time used and mileage detected by the memory unit 35, and the current speed of the bicycle.

The advantages of the present invention are that the force assistance unit 32 calculates the electric power consumption value 121, the acceleration value 141 and the heart rate signal 21 to create the motor output adjustment signal 321 which represents the acceleration speed of the bicycle 1 that is purely caused by the human power. The motor output adjustment signal 321 is sent to the micro controller 13 to control output of the battery 12 and the motor 11 so as to provide a proper assistance force to the rider 4 to operate the bicycle 1 more efficiently.

The output of the battery 12 is adjusted according to the inclination to ensure that the power from the battery 12 is sufficient to let the rider 4 to operate the bicycle 1 to its destination by the results that the force assistance unit 32 calculates the altitude from the GPS navigation chip 33 and calculates the inclination of the travel route, and the force assistance unit 32 calculates the electric power consumption value 121, the acceleration value 141 and the heart rate signal 21 to create the motor output adjustment signal 321.

While we have shown and described the embodiment in accordance with the present invention, it should be clear to those skilled in the art that further embodiments may be made without departing from the scope of the present invention.

What is claimed is:

1. A control device for an electric bicycle, comprising:
a motor, a battery, a micro controller, an acceleration detector and a transmitter electrically connected to each other, the micro controller controlling the battery to provide power to the motor of the electric bicycle, and monitoring the power output from the battery and defining an electric power consumption value, the acceleration detector detecting the acceleration speed of the bicycle and creating an acceleration value, the transmitter transmitting the electric power consumption value and the acceleration value to a computer, and
the computer having a wireless receiving/emitting unit and a force assistance unit, the wireless receiving/emitting unit receiving the electric power consumption value and the acceleration value, the force assistance unit calculating the electric power consumption value and the acceleration value, and creating a motor output adjustment signal, the wireless receiving/emitting unit and the transmitter sending the motor output adjustment signal to the micro controller which adjusts the battery to output power to the motor so as to be adapted to change or adjust assistance force to the rider who operates the electric bicycle.

2. The control device for an electric bicycle as claimed in claim 1 further comprising a physical-condition monitor which is adapted to be attached to the rider, the physical-condition monitor is adapted to monitor physical condition of the rider and creating a heart rate signal which is sent to the computer wirelessly, the force assistance unit calculates the electric power consumption value, the acceleration value and the heart rate signal and creates the motor output adjustment signal, the wireless receiving/emitting unit and the transmitter send the motor output adjustment signal to the micro controller which adjusts the battery to output power to the motor so as to be adapted to change or adjust assistance force to the rider who operates the electric bicycle.

3. The control device for an electric bicycle as claimed in claim 2, wherein the computer is a laptop, a smart phone or a tablet computer, and includes a Global Positioning System navigation chip (GPS navigation chip), a touch screen, a memory unit and a setting unit, the GPS navigation chip is adapted to receive signals via satellites to obtain current location and altitude information, the setting unit sets a travel route, the touch screen is adapted to display the travel route and current locations, the memory unit records the electric power consumption value, the acceleration value and the heart rate signal from a start point of the travel route, the force assistance unit calculates the altitude from the GPS navigation chip and calculates inclination of the travel route, and the force assistance unit calculates the electric power consumption value, the acceleration value and the heart rate signal to create the motor output adjustment signal.

4. The control device for an electric bicycle as claimed in claim 1, wherein the acceleration detector is a gyro.

5. The control device for an electric bicycle as claimed in claim 2, wherein the physical-condition monitor supports a wireless ring or a heart rate band.
